(19) United States
(12) Patent Application Publication
(10) Pub. No.: US 2008/0120767 A1
(43) Pub. Date: May 29, 2008

(54) FACE-PIECE FOR DIVING GOGGLES

(76) Inventor: Giovanni GAROFALO, Rapallo (IT)

Correspondence Address: STITES & HARBISON PLLC, 1199 NORTH FAIRFAX STREET, SUITE 900, ALEXANDRIA, VA, 22314, US

(21) Appl. No.: 11/939,202

(22) Filed: Nov. 13, 2007

(30) Foreign Application Priority Data
Nov. 24, 2006 (IT) GE2006U000023

(51) Int. Cl. A61F 9/02

(52) U.S. Cl. 2/428

(57) ABSTRACT

Face-piece for diving goggles, of the type comprising a face-piece made of an elastomer material such as rubber, synthetic rubber, silicone or the like and provided with a shaped recessed seat for the diver's nose. The outer side of the face-piece of the goggles, on both sides of the said shaped recessed seat for the nose, is provided with a series of projections which extend laterally.

### BACKGROUND OF THE INVENTION

1. Field of the Invention

The present invention relates to diving goggles and more particularly to the face-pieces of diving goggles.

2. Description of Related Art

As is known, the face-pieces of the goggles comprise a seat for the diver's nose, which, in the case of goggles of the double-lens type, is situated between two rigid frames for fixing the lenses of the goggles, while in the case of single-lens goggles, it is situated between the central uprights—shaped substantially in the form of an inverted V—of the single frame which supports the lens.

One of the problems which these face-pieces pose is that, when the diver turns his/her head to one side or the other, the lens-fixing frames make contact with the diver's nostrils, causing considerable discomfort for the diver.

### BRIEF SUMMARY OF THE INVENTION

The main object of the present invention is to overcome said drawback of the known goggles.

This object is achieved in accordance with the main characteristic feature of the present invention by providing on the outer side of the face-piece of the goggles, on the sides of the recess seating the diver's nose, a series of small ribs which extend into the vicinity of the lens support frame or frames.

Advantageously these ribs are formed as one piece with the material of the face-piece of the goggles.

### BRIEF DESCRIPTION OF THE SEVERAL VIEWS OF THE DRAWINGS

Further objects, characteristic features and advantages of the present invention will emerge more clearly from the following description of a preferred embodiment thereof provided with reference to the accompanying drawings in which:

FIG. 1 is a perspective view of a face-piece of diving goggles of the double-lens type according to the invention, and

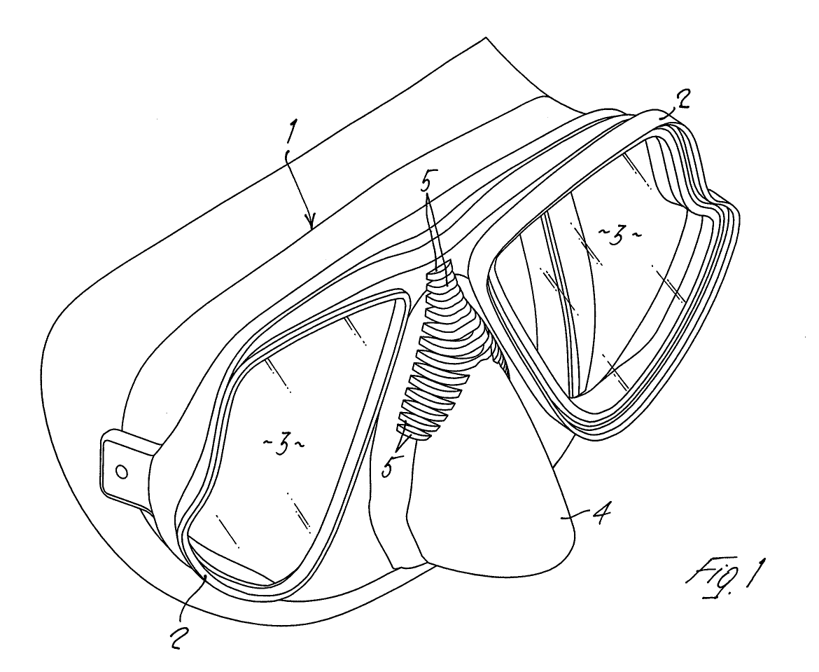

FIG. 2 is a front view thereof.

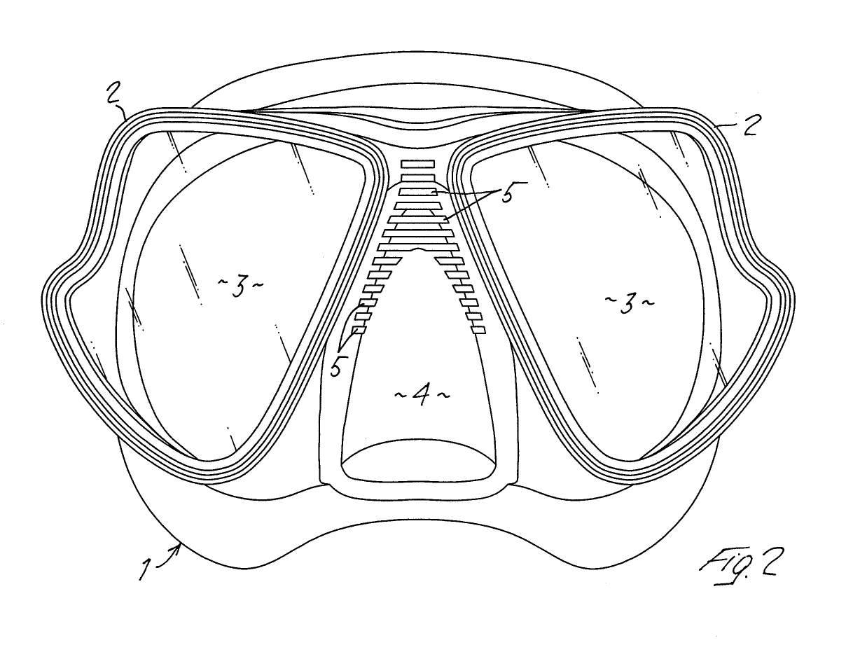

### DETAILED DESCRIPTION OF THE INVENTION

With reference to the drawings, the goggles shown comprise, in a usual manner, a face-piece 1 which is made of an elastomer material such as rubber, synthetic rubber, silicone or the like and has two openings in the region of the diver's eyes which are surrounded by two frames 2 which are generally made of rigid plastic material and to which the lenses 3 are fastened. In the zone between the two eye frames 2 the face-piece 1 has a seat 4 for the diver's nose.

In order to prevent, following sideways movements of the diver's head, the frames 2 from striking the diver's nostrils, the periphery of the seat 4 for the nose is provided with a whole series of ribs 5 which are made of the same material as the face-piece 1 and prevent the frames 2 from striking the diver's nostrils or cushion any impacts caused by them.

The above is also applicable in the case of single-lens goggles which have a single frame which, along its upper section, extends substantially in a straight line along the user's forehead and which, along its bottom section, surrounds the nose seat 4 with a frame element having a profile substantially in the form of an overturned V.

Obviously, the present invention is not limited to the embodiment illustrated and described, but comprises all those modifications which fall within the scope of the inventive idea.

Thus, for example, the ribs 5 may be replaced by other barrier elements such as small studs or the like.

1. Face-piece for diving goggles, of the type comprising a face-piece made of an elastomer material such as rubber, synthetic rubber, silicone or the like and provided with a shaped recessed seat for the diver's nose, characterized in that the outer side of the face-piece of the goggles, on both sides of the said shaped recessed seat for the nose, is provided with a series of projections which extend laterally.

2. Face-piece according to claim 1, characterized in that said projections are in the form of small ribs which extend transversely with respect to the said seat for the diver's nose.

3. Face-piece according to claim 2, in which said projections are arranged on the periphery of the said seat for the diver's nose.

4. Face-piece according to claim 1, suitable for double-lens or single-lens goggles.
